(19) United States
(12) Patent Application Publication
(10) Pub. No.: US 2007/0071688 A1
(43) Pub. Date: Mar. 29, 2007

(54) Pharmaceutical compositions for transdermal administration of anti-inflammatory agents

(75) Inventors: Brigitte Illel, Montpellier (FR); Jean-Pierre Vergnaud, Montpellier (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: sanofi-aventis, PARIS (FR)

(21) Appl. No.: 11/549,168

(22) Filed: Oct. 13, 2006

Related U.S. Application Data
(63) Continuation of application No. 10/275,801, filed on Nov. 8, 2002, now abandoned.

(30) Foreign Application Priority Data
May 12, 2000 (FR) 00/06048

(51) Int. Cl. A61K 9/12

(52) U.S. Cl. 424/45

(57) ABSTRACT

A pharmaceutical composition for transdermal administration comprising a polymeric release matrix capable of forming a supple film after drying, selected among cellulose polymers or copolymers; an active principle selected among the group of non-steroid anti-inflammatory agents comprising at least one carboxylic or metal carboxylate group; a transcutaneous absorption promoter; water; and a physiologically acceptable non-aqueous solvent capable of dissolving the release matrix, the active principle and the transcutaneous absorption promoter and to be rapidly eliminated by evaporation on contact with the skin.

The present invention relates generally to a novel pharmaceutical composition for transdermal administration.

More specifically, the invention relates to a pharmaceutical composition for transdermal administration of nonsteroidal anti-inflammatory agents such as ibuprofen, the composition being capable of forming a supple film after drying on the skin.

The transdermal administration of medicinal active principles is an appealing technique, since it is noninvasive, which has undoubted advantages such as the absence of gastrointestinal side effects or of alterations of the active substance by the enzymes of the liver.

However, to be effective, this technique must allow a transcutaneous penetration of the medicinal product over a prolonged period and in a sufficient amount to reach levels in the plasma that are compatible with a therapeutic treatment.

To date, various systems or devices for this type of administration have been proposed, to allow the introduction, into the blood stream, of controlled doses of medicinal substances.

For example, the transdermal administration device commonly known as a “patch”, consisting of a reservoir formed of synthetic plastic materials containing the active principle, is known. This reservoir may be coated, on its face in contact with the skin, with a microporous membrane whose permeability to the active substance regulates its diffusion and consequently its dosage.

Despite the genuine possibilities offered by this device, other systems may be preferred to it. The reason for this is that it is known that the patch can become detached from the skin and, moreover, it often has an unattractive appearance.

Gels containing various active principles have also been proposed. However, this pharmaceutical form may have certain drawbacks in use, generally a sticky feel that the patient finds unpleasant, and also difficulty in controlling the dose of active principle administered and difficulty in controlling the area of coverage.

Other systems have also been reported, which aid the transdermal administration of medicinal principles.

In this respect, mention may be made of sprayable compositions especially comprising polymers capable of forming a film on contact with the skin and of releasing the active principle for transcutaneous administration. Compositions of this type, which are described for example in patent EP 0 319 555, comprise an active principle, a polymer matrix forming a supple film after drying, a solvent controlling the release of the active substance, namely a sorbitan macrogollaurate, a paraffin, a medium-chain fatty acid diglyceride or triglyceride or propylene carbonate and also a solvent, for the matrix, capable of evaporating on the skin, and finally a propellant for spraying this composition contained in a suitable device.

However, a matrix consisting of ethylcellulose is not recommended therein on account of its tendency to block the spraying system.

In addition, compositions such as those proposed by the abovementioned patent, characterized by the presence of a propellant gas, for example a halohydrocarbon, are coming under increasing debate as a result of the potential risks they are liable to pose to the environment.

What is more, due to the presence of polymethacrylic derivatives, the compositions of patent EP 0 319 555 give off a characteristic odor that is relatively unpleasant to the patient or people in his vicinity.

Other pharmaceutical compositions for topical administration containing an active principle, a solvent and various other ingredients are also known.

Examples that may be mentioned include patent EP 55396, which describes antimycotic compositions formed:
from a cellulose ether
from 2% to 10% of a spreading agent such as isopropyl myristate or isopropyl palmitate
from 1% to 8% of a solubilizing agent
from 0.05% to 1% of an active principle, and
from a solvent such as isopropanol.

However, although these compositions may be used for dermatological topical applications, they are found to be entirely unsuitable for application by spraying, even after adding from 10% to 40% of a propellant gas as recommended, since they appear too viscous and liable to give rise to various drawbacks such as blocking of the spraying device.

Mention may also be made of patent EP 319 964 describing antifungal compositions capable of forming a film comprising:
from 0.1% to 1.5% tolnaphthalate
from 10% to 20% of a dimethylaminoethyl methacrylate/methacrylate copolymer
from 0.5% to 10% of a fatty acid ester
a solvent of alcohol type and optionally from 0.1% to 5% of a cellulose derivative.

This composition does not appear to be suitable for spraying either. In addition, as already stated above, the presence of methacrylic derivatives gives it an unacceptable odor.

Moreover, mention may be made of patent EP 289 900 which relates to antibacterial compositions for topical use, comprising:
from 0.5% to 10% of an antibacterial active principle
from 1% to 30% of a water-insoluble polymer, especially ethylcellulose or a polyvinylpyrrolidone copolymer
from 0.5% to 40% of a plasticizer, generally an essential oil, that also acts as a transcutaneous absorption promoter
from 50% to 95% of a solvent such as ethanol.

As is known, essential oils predominantly consist of terpene derivatives.

In the context of the invention, a composition similar to that described in said patent has been investigated, especially containing estradiol as active principle and limonene, which is a terpene, as transdermal absorption promoter. However, such a composition gave only very low transcutaneous diffusion flows of this active principle.

Furthermore, mention may be made of patent application EP 0 581 587, which describes a pharmaceutical composition concerning estradiol, hydroxypropylcellulose, isopropyl myristate, water and ethanol.

However, this composition, which contains 15% by weight of hydroxypropylcellulose, might not be sprayable on account of the excessively large amount of this cellulose derivative. This composition is moreover in the form of a gel.

Finally, mention may be made of patent application WO 96/30000, which describes film-forming compositions for transdermal administration, comprising:
up to 6% of a matrix formed from cellulosic polymers or copolymers
from 0.1% to 20% by weight of an active principle
from 15% to 30% by weight of a transcutaneous absorption promoter chosen from aliphatic fatty acids or aliphatic fatty alcohols
from 44% to 84.9% of a nonaqueous solvent.

Examples have been described in said patent application which illustrate compositions containing ethylcellulose as matrix, ibuprofen as active principle, 2-ethylhexyl 2-ethylhexanoate as transcutaneous absorption promoter and ethanol as nonaqueous solvent.

However, these compositions are of precarious stability over time, which makes it difficult to use them a certain number of years after their manufacture, especially after three years.

The search for a composition allowing the transdermal diffusion of medicinal active principles, especially ibuprofen, at levels that are compatible with a therapeutic treatment, while at the same time being free of the drawbacks previously reported, remains of major interest.

In the context of the present invention, preliminary trials were carried out directed essentially toward studying the solubility of ibuprofen in various solvents or components. It has thus been possible to confirm that this active principle is soluble in fatty acid ester/alcohol mixtures such as those described in patent application WO 96/30000, especially 2-ethylhexyl 2-ethylhexanoate/ethanol or isopropanol mixtures, so as to form homogeneous solutions.

However, ibuprofen was found to be entirely water-insoluble.

Now, it has been discovered, surprisingly, that the addition of water to the fatty acid/alcohol mixtures of the abovementioned patent application, in particular the addition of water to 2-ethylhexyl 2-ethylhexanoate/ethanol or isopropanol mixtures, makes it possible to increase the solubility of ibuprofen in these fatty acid ester/alcohol mixtures and to provide pharmaceutical compositions that are particularly stable and free of the drawbacks mentioned above, while at the same time being capable of delivering this active principle into the blood stream at levels largely reaching therapeutic thresholds.

Thus, the main subject of the invention is a pharmaceutical composition for transdermal administration, comprising:
a polymeric release matrix capable of forming a supple film after drying
an active principle
a promoter of transcutaneous absorption of the active principle
water
at least one physiologically acceptable nonaqueous solvent capable of dissolving the release matrix, the active principle and the transcutaneous absorption promoter, and also of being rapidly eliminated by evaporation on contact with the skin.

In the present context, both in the description and in the claims, the term “active principle” means either a medicinal substance intended, after administration, to bring about a preventive or therapeutic response, or a combination of two or more substances of this type.

The polymer matrix is generally chosen from polymer or copolymer substances capable both of forming a supple film after the solvent has evaporated off, and of releasing the active principle. This matrix will be chosen from substances that are soluble in the physiologically acceptable solvent(s) so as to form a homogeneous solution.

In addition, this matrix is present at a concentration not exceeding 6% of the weight of the composition according to the invention, for example from 0.5% to 2%. Preferably, from 0.5% to 1% by weight of matrix is used, especially 0.5%.

The polymers or copolymers capable of satisfying the above criteria will be chosen from cellulosic polymers and copolymers, especially since they have suitable abrasion resistance and mechanical stability after drying. For this reason, cellulose matrices of this type may be rinsed with water without any fear of deterioration.

As examples of such cellulosic polymers or copolymers that may be used in the compositions of the invention, mention may be made of ethylcellulose, cellulose acetate butyrate, cellulose acetate propionate or a grafted or ungrafted hydroxypropylmethylcellulose, such as hydroxypropylmethylcellulose acetate succinate.

However, ethylcellulose is the preferred cellulosic polymer and, consequently, the polymeric release matrix of choice for the formation of a supple film on contact with the skin.

The active principle will be chosen from the group of nonsteroidal anti-inflammatory agents, i.e. analgesic, antipyretic and anti-inflammatory medicinal substances comprising at least one carboxylic or metal carboxylate group such as an alkali metal carboxylate, these medicinal substances being soluble in the physiologically acceptable solvent(s) and capable of continuously crossing the epidermis and the dermis with a flow that is sufficient to reach therapeutically effective levels.

Such substances will also be selected from those that show a large physiological effect at low plasmatic levels.

Examples that may be mentioned include ibuprofen, alminoprofen, benoxaprofen, indoprofen, fenoprofen, flurbiprofen, ketoprofen, tiaprofenic acid, acetylsalicylic acid, salicylic acid, naproxen, clonixin, niflumic acid, indomethacin, mefenamic acid, alclofenac, diclofenac, etodolac, sulindac, tianafac and flufenamic acid.

These medicinal active principles, comprising ibuprofen, will be incorporated into the compositions of the invention at concentrations not exceeding 15% of the weight of these compositions, especially from 1% to 15% and preferably from 3% to 10%, given that each active principle will be introduced at individualized concentrations known to those skilled in the art for a transdermal administration or adapted for this administration route.

For example, ibuprofen may feature among the compositions of the invention in a proportion of from 4% to 10% of the weight of this composition, especially from 4% to 5% and preferably 5%.

In order to achieve a transcutaneous diffusion that is sufficient to obtain the therapeutic efficacy of the active principle, a transcutaneous absorption promoter whose concentration will not exceed 40% by weight of the composition is combined with the polymer matrix and with this active principle. Said promoter is included in the compositions of the invention advantageously in a proportion of from 10% to 30% of the weight of this composition, preferably from 15% to 25%, for example 20%.

In order to be effective, the transcutaneous absorption promoter under consideration must be capable of temporarily disorganizing the skin barrier so as to increase the permeability of the skin without irritating it, while at the same time promoting the diffusion of the active principle chosen according to sufficient kinetics and a sufficient concentration that may be maintained for a certain time.

This transcutaneous absorption promoter will be selected from substances that are soluble in physiologically acceptable nonaqueous solvents, capable of evaporating rapidly on contact with the skin.

Said promoter will preferably be chosen from the following compounds, which have the required degree of solubility in the nonaqueous solvent(s) under consideration and which combine the best qualities reported above, i.e. from:
aliphatic fatty acid esters, essentially esters containing in total from 10 to 30 carbon atoms optionally substituted with one or two hydroxyl, carboxylic or C₁-C₄ acyloxy groups such as acetoxy, or optionally interrupted with one or two ethylenic bonds or with one or two ether oxygens,
aliphatic fatty alcohols, essentially C₁₀-C₃₀ alcohols optionally substituted with one or two hydroxyl, carboxylic or C₁-C₄ acyloxy groups such as acetoxy or optionally interrupted by one or two ethylenic bonds or with one or two ether oxygens.

Absorption promoters that are particularly preferred, which may be selected from the aliphatic fatty acid esters and aliphatic fatty alcohols mentioned above, are reported below, namely:

a) aliphatic fatty acid esters of general formula: 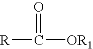
in which R represents a linear or branched C₂-C₁₇ alkyl or alkenyl group optionally substituted with a hydroxyl, carboxylic or C₁-C₄ acyloxy group and R₁ represents a linear or branched C₃-C₈ alkyl group optionally substituted with one or two hydroxyl groups such as, for example, an isopropyl, 2-ethylhexyl or 1,2-dihydroxyethyl group or R₁ represents a —CH₂—CH₂—O—(CH₂)₂—O—CH₂—CH₃ group, the aliphatic fatty acid ester containing a minimum of 10 carbon atoms and a maximum of 2 hydroxyl groups,
b) aliphatic fatty alcohols of general formula:
   R₂—OH
in which R₂ represents a C₁₀-C₂₀ alkyl group.

As particular compounds that have shown the best potential for promoting the transcutaneous absorption of active principles, especially ibuprofen, mention may be made of:
2-ethylhexyl 2-ethylhexanoate
isopropyl myristate
diethylene glycol monoethyl ether myristate
isopropyl palmitate
2-octyldodecanol
2-ethylhexyl undecylenate
2-ethylhexyl succinate
2-ethylhexyl 12-hydroxystearate
2-ethylhexyl 12-acetoxystearate
glyceryl isostearate
hexyl laurate.

2-ethylhexyl 2-ethylhexanoate represents the preferred absorption promoter, especially for transdermal compositions according to the invention whose active principle is ibuprofen.

As regards water, it is introduced into the compositions of the invention at a concentration that is compatible with the amount of cellulosic polymers or copolymers chosen and with an acceptable drying time of the composition sprayed onto the skin.

Generally, this water concentration will not exceed 30% of the weight of the total composition. It will preferably be from 3% to 10% by weight especially from 3% to 5%.

As regards the physiologically acceptable nonaqueous solvent(s) capable of dissolving the release matrix, the active principle and the transcutaneous absorption promoter, it (they) will be chosen from compounds with a boiling point that is relatively low, i.e. below 100° C. at atmospheric pressure, so that they can be eliminated rapidly by evaporation on contact with the skin and thereby help to form a film by drying, without, however, causing any local irritation.

Such physiologically acceptable solvents are generally used in an amount that is sufficient to reach 100% of the weight of the final composition usually at concentrations greater than 50% of the weight of this composition. In addition, they may be selected from volatile compounds such as dichloromethane, ethanol, isopropanol and ethyl acetate.

Ethanol and isopropanol are solvents of choice. However, isopropanol is a preferred solvent according to the invention.

Consequently, according to one of its particular aspects, the invention relates to a transdermal composition comprising, by weight:
from 0.5% to 2% of a polymeric release matrix capable of forming a supple film after drying, chosen from cellulosic polymers or copolymers such as ethylcellulose
from 3% to 10% of an active principle, in particular from 3% to 6% of ibuprofen
from 10% to 30% of a promoter of transcutaneous absorption of the active principle, in particular from 15% to 25% of a fatty acid ester or of a fatty alcohol chosen from:
   2-ethylhexyl 2-ethylhexanoate
   isopropyl myristate
   diethylene glycol monoethyl ether myristate
   isopropyl palmitate
   2-octyldodecanol
   2-ethylhexyl undecylenate
   2-ethylhexyl succinate
   2-ethylhexyl 12-hydroxystearate
   2-ethylhexyl 12-acetoxystearate
   glyceryl isostearate
   hexyl laurate
from 3% to 10% of water
a sufficient amount, to reach 100% of the weight of the composition, of at least one physiologically acceptable nonaqueous solvent capable of dissolving the release matrix, the active principle and the transcutaneous absorption promoter, and also of being rapidly eliminated by evaporation on contact with the skin, in particular ethanol or isopropanol.

If necessary, the compositions of the invention will also comprise an aromatizing fraction consisting of one or more aromatizing compounds such as camphor or, preferably, levomenthol.

This aromatizing fraction will be used at concentrations not exceeding 5% by weight of the composition, especially for the sensation of freshness it may afford on the skin. By way of example, levomenthol may be present in the compositions of the invention at a concentration of 2% by weight of the composition.

Characteristics and advantages of the compositions of the invention will emerge in the light of the description below.

Solubility Tests

Comparative tests were carried out in order to determine the saturation solubility of ibuprofen in various components of the transdermal formulation of the invention in the absence of matrix, namely water, 2-ethylhexyl 2-ethylhexanoate as transcutaneous absorption promoter, referred to hereinbelow as “promoter”, and isopropanol as nonaqueous solvent.

These various components were used alone or as homogeneous mixtures forming a clear solution, and the solubility measurements were performed at 30° C., after 24 hours of magnetic stirring and after filtration through a 0.22 μm filter.

The following results were obtained:

a) Components alone

| | | | Ibuprofen |
|---|---|---|---|
| | | | saturating |
| Water | Isopropanol | Promoter | concentration |
| (% m/m) | (% m/m) | (% m/m) | (mg/ml) |
| 100 | — | — | 0.069 |
| — | 100 | — | 441 |
| — | — | 100 | 192 |

b) Components as binary or ternary mixtures

| | | | Ibuprofen |
|---|---|---|---|
| | | | saturating |
| Water | Isopropanol | Promoter | concentration |
| (% m/m) | (% m/m) | (% m/m) | (mg/ml) |
| — | 68.21 | 31.79 | 403 |
| 4.38 | 68.16 | 27.46 | 431 |
| 8.54 | 68.23 | 23.23 | 436 |
| — | 89.31 | 10.69 | 424 |
| 4.33 | 85 | 10.67 | 451 |
| 8.59 | 80.7 | 10.69 | 448 |

These results show that:
if a weight fraction of isopropanol is replaced with promoter, a component in which ibuprofen is less than half as soluble, the solubility of this active principle in these homogeneous binary mixtures is reduced;
if a weight fraction of isopropanol or of promoter in their homogeneous binary mixtures is replaced with water, a component in which ibuprofen is virtually insoluble, the solubility of this active principle in these homogeneous ternary mixtures is, on the other hand, increased.

All the substances forming part of the compositions of the invention for transdermal administration are known products or products that may be prepared by known methods, some of these products being commercially available.

These compositions according to the invention may be prepared, conventionally, by mixing the various constituents in proportions chosen so as to form homogeneous mixtures.

For example, the transcutaneous absorption promoter can be dissolved, with stirring, in the physiologically acceptable solvent(s) containing water, and the active principle and finally the release matrix can then be added.

The transdermal compositions of the invention thus obtained may be applied by any means to a given area of skin, especially and preferably by direct spraying using a metering pump of known and commercial type without the aid of a propellant such as a compressed or liquefied gas.

Since the addition of water and of cellulosic polymer or copolymer such as ethylcellulose to a mixture of active principle such as ibuprofen, transcutaneous absorption promoter such as 2-ethylhexyl 2-ethylhexanoate and an alcohol such as isopropanol increases the surface tension of the mixture, care will be taken to ensure that the surface tension of the final composition is compatible with efficient spraying.

Although the prior art asserts the contrary, it has been found, surprisingly, that a release matrix formed from ethylcellulose, which is nevertheless at the concentrations recommended in the context of the invention, does not cause any obstruction by sticking at the outlet of the endpiece of the spraying head, such that the compositions of the invention may be sprayed without the need for a propellant gas and without any fear of deterioration of the spray container.

However, if so desired, the compositions of the invention may be administered by spraying using a container equipped with a metering valve, also containing a compressed propellant gas such as nitrogen or nitrous oxide, or a liquefied propellant gas such as butane.

The film-forming compositions of the invention have unquestionable advantages since they are capable of bringing about the sufficient transcutaneous diffusion of a nonsteroidal anti-inflammatory active principle, for example ibuprofen, so as to produce therapeutic levels, in contrast with the levels released by the known compositions and known matrices for transdermal compositions, such as those described, for example, in patent EP 0 319 555.

Moreover, the compositions of the invention have been found to have considerable stability, in contrast with compositions given as examples in patent application WO 96/30000. Specifically, three years after their manufacture, compositions of the invention containing, for example, ibuprofen as active principle, in particular the preferred composition below:

| | weight % |
|---|---|
| Ethylcellulose | 0.5 |
| Ibuprofen | 5 |
| 2-Ethylhexyl 2-ethylhexanoate | 20 |
| Water | 4 |
| Isopropanol | 70.5 |

should have less than 5% by weight of ibuprofen conversion products, whereas the contents of the corresponding products derived from ibuprofen that are present in the prior compositions should be significantly higher.

In addition, the compositions according to the invention, while being free of any unpleasant odor, spread out to a uniform film over the selected area of skin and, to this end, do not necessarily require environmentally unfriendly propellant gases.

These films are sufficiently supple and abrasion-resistant to avoid any deterioration on the skin of the patient, and show better tolerability than the known transdermal devices since, on account of their thinness and the absence of any covering, the gaseous and aqueous exchanges with the exterior are not necessarily disrupted.

Finally, the compositions of the invention, in the form of a supple film, are more comfortable for the patient than a transdermal patch and, by virtue of their transparency, are totally invisible.

The following nonlimiting examples illustrate the preparation of compositions of the invention.

### EXAMPLE 1

### Transdermal Composition Containing Ibuprofen

100 g of a transdermal composition having the formulation below are prepared:

| | weight % |
|---|---|
| Ethylcellulose 6 mPa.sec | 0.5% |
| Ibuprofen | 5% |
| 2-Ethylhexyl 2-ethylhexanoate | 20% |
| Isopropanol | 70.5% |
| Water | 4% |

by rapidly mixing, with magnetic stirring, 70.5 g of isopropanol, 4 g of water and 20 g of 2-ethylhexyl 2-ethylhexanoate.

5 g of ibuprofen are then added portionwise to the mixture obtained and, once completely dissolved (5 minutes), 0.5 g of ethylcellulose 6 mPa·sec is then introduced, with vigorous stirring, so as to avoid the formation of lumps. The final solution obtained is homogeneous and slightly opalescent.

For the purpose of administration by spraying, cans are filled with 50 ml of the solution obtained above and are equipped with a crimp-on vasopump comprising a press-button.

The pump is actuated twice to prime it before its first use.

### EXAMPLES 2 TO 8

### Transdermal Compositions Containing Ibuprofen

Using the same process as in Example 1, the transdermal compositions having the formulations below were prepared:

| | | weight % |
|---|---|---|
| EX. 2 | Ethylcellulose | 0.5% |
| | Ibuprofen | 5% |
| | Isopropyl myristate | 20% |
| | Isopropanol | 70.5% |
| | Water | 4% |
| EX. 3 | Ethylcellulose | 2% |
| | Ibuprofen | 5% |
| | 2-Ethylhexyl 2-ethylhexanoate | 20% |
| | Isopropanol | 69% |
| | Water | 4% |
| EX. 4 | Ethylcellulose | 0.5% |
| | Ibuprofen | 10% |
| | 2-Ethylhexyl 2-ethylhexanoate | 20% |
| | Isopropanol | 65.5% |
| | Water | 4% |
| EX. 5 | Ethylcellulose | 0.5% |
| | Ibuprofen | 5% |
| | 2-Ethylhexyl succinate | 20% |
| | Isopropanol | 70.5% |
| | Water | 4% |
| EX. 6 | Ethylcellulose | 0.5% |
| | Acetylsalicylic acid | 5% |
| | 2-Ethylhexyl 2-ethylhexanoate | 20% |
| | Isopropanol | 70.5% |
| | Water | 4% |
| EX. 7 | Ethylcellulose | 0.5% |
| | Acetylsalicylic acid | 5% |
| | 2-Ethylhexyl succinate | 20% |
| | Isopropanol | 70.5% |
| | Water | 4% |
| EX. 8 | Ethylcellulose | 0.5% |
| | Ibuprofen | 5% |
| | 2-Ethylhexyl 2-ethylhexanoate | 20% |
| | Water | 4% |
| | Isopropanol | 68.5% |
| | Levomenthol | 2% |

1. A sprayable pharmaceutical composition for transdermal administration comprising:
a polymeric release matrix capable of forming a supple film after drying, chosen from cellulosic polymers and copolymers, this matrix being present in a proportion of from 0.5% to 2% of the weight of the composition,
an active principle chosen from the group of nonsteroidal anti-inflammatory agents comprising at least one carboxylic or metal carboxylate group,
a promoter of transcutaneous absorption of the active principle,
water, and
a physiologically acceptable nonaqueous solvent capable of dissolving the release matrix, the active principle and the transcutaneous absorption promoter, and also of being rapidly eliminated by evaporation on contact with the skin.

2. A sprayable pharmaceutical composition according to claim 1 wherein the polymeric release matrix is present in a proportion of from 0.5% to 1% of the weight of the composition.

3. A sprayable pharmaceutical composition according to claim 1 wherein the active principle is present at a concentration not exceeding 15% of the weight of the composition.

4. A sprayable pharmaceutical composition according to claim 3 wherein the active principle is present in a proportion of from 3% to 10% of the weight of the composition.

5. A sprayable pharmaceutical composition according to claim 1 wherein the transcutaneous absorption promoter is present at a concentration not exceeding 40% of the weight of the composition.

6. A sprayable pharmaceutical composition according to claim 5 wherein the transcutaneous absorption promoter is present in a proportion of from 10% to 30% of the weight of the composition.

7. A sprayable pharmaceutical composition according to claim 6 wherein the transcutaneous absorption promoter is present in a proportion of from 15% to 25% of the weight of the composition.

8. A sprayable pharmaceutical composition according to claim 1 wherein the water is present at a concentration not exceeding 30% of the weight of the composition.

9. A sprayable pharmaceutical composition according to claim 8 wherein the water is present in a proportion of from 3% to 10% of the weight of the composition.

10. A sprayable pharmaceutical composition according to claim 1 wherein the physiologically acceptable nonaqueous solvent is present in an amount that is sufficient to reach 100% of the weight of the composition.

11. A sprayable pharmaceutical composition according to claim 2 wherein the polymeric release matrix is ethylcellulose, cellulose acetate butyrate, cellulose acetate propionate or a grafted or ungrafted hydroxypropylmethylcellulose.

12. A sprayable pharmaceutical composition according to claim 11 wherein the polymeric release matrix is ethylcellulose.

13. A sprayable pharmaceutical composition according to claim 4 wherein the active principle is chosen from ibuprofen, alminoprofen, benoxaprofen, indoprofen, fenoprofen, flurbiprofen, tiaprofenic acid, acetylsalicylic acid, salicylic acid, naproxen, clonixin, niflumic acid, indomethacin, mefenamic acid, alclofenac, diclofenac, etodolac, sulindac, tianafac and flufenamic acid.

14. A sprayable pharmaceutical composition according to claim 13 wherein the ibuprofen is present in a proportion of from 4% to 10% of the weight of the composition.

15. A sprayable pharmaceutical composition according to claim 14 wherein the ibuprofen is present in a proportion of from 4% to 5% of the weight of the composition.

16. A sprayable pharmaceutical composition according to claim 7 wherein the transcutaneous absorption promoter is chosen from:
an aliphatic fatty acid ester, which is soluble in the physiologically acceptable nonaqueous solvent(s), containing in total from 10 to 30 carbon atoms and being optionally substituted with one or two hydroxyl, carboxylic or C₁-C₄ acyloxy groups or optionally interrupted with one or two ethylenic bonds or with one or two ether oxygens, and
a C₁₀-C₃₀ aliphatic fatty alcohol, which is soluble in the physiologically acceptable nonaqueous solvent(s), and optionally substituted with one or two hydroxyl, carboxylic or C₁-C₄ acyloxy groups or optionally interrupted with one or two ethylenic bonds or with one or two ether oxygens.

17. A sprayable pharmaceutical composition according to claim 16 wherein the transcutaneous absorption promoter is chosen from:
an aliphatic fatty acid ester that is soluble in the physiologically acceptable nonaqueous solvent(s) and of general formula: 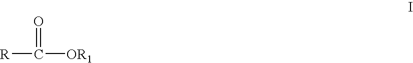
in which R represents a linear or branched C₂-C₁₇ alkyl or alkenyl group optionally substituted with a hydroxyl, carboxylic or C₁-C₄ acyloxy group and R₁ represents a linear or branched C₃-C₈ alkyl group optionally substituted with one or two hydroxyl groups or R₁ represents a —CH₂—CH₂—O—(CH₂)₂—O—CH₂—CH₃ group, the aliphatic fatty acid ester containing a minimum of 10 carbon atoms and a maximum of 2 hydroxyl groups, and
an aliphatic fatty alcohol, which is soluble in the physiologically acceptable nonaqueous solvent(s) and of general formula:
R₂—OH II
in which R₂ represents a C₁₀-C₂₀ alkyl group.

18. A sprayable pharmaceutical composition according to claim 17 wherein R₁ represents an isopropyl, 2-ethylhexyl or 1,2-dihydroxyethyl group.

19. A sprayable pharmaceutical composition according to claim 18 wherein the transcutaneous absorption promoter is chosen from:
2-ethylhexyl 2-ethylhexanoate,
isopropyl myristate,
diethylene glycol monoethyl ether myristate,
isopropyl palmitate,
2-octyldodecanol,
2-ethylhexyl undecylenate,
2-ethylhexyl succinate,
2-ethylhexyl 12-hydroxystearate,
2-ethylhexyl 12-acetoxystearate,
glyceryl isostearate, and
hexyl laurate.

20. A sprayable pharmaceutical composition according to claim 19 wherein the transcutaneous absorption promoter is 2-ethylhexyl 2-ethylhexanoate.

21. A sprayable pharmaceutical composition according to claim 10 wherein the physiologically acceptable nonaqueous solvent is a compound with a boiling point below 100° C. at atmospheric pressure.

22. A sprayable pharmaceutical composition according to claim 21 wherein the compound with a boiling point below 100° C. is dichloromethane, ethanol, isopropanol or ethyl acetate.

23. A sprayable pharmaceutical composition according to claim 22 wherein the compound with a boiling point below 100° C. is isopropanol.

24. A sprayable pharmaceutical composition for transdermal administration which comprises, by weight:
a) from 0.5% to 2% of a polymeric release matrix capable of forming a supple film after drying, chosen from cellulosic polymers or copolymers,
b) from 3% to 10% of an active principle chosen from the group of nonsteroidal anti-inflammatory agents containing at least one carboxylic or metal carboxylate group,
c) from 10% to 30% of a promoter of transcutaneous absorption of the active principle chosen from:
2-ethylhexyl 2-ethylhexanoate,
isopropyl myristate,
diethylene glycol monoethyl ether myristate,
isopropyl palmitate,
2-octyldodecanol,
2-ethylhexyl undecylenate,
2-ethylhexyl succinate,
2-ethylhexyl 12-hydroxystearate,
hexyl laurate,
glyceryl isostearate, and
2-ethylhexyl 12-acetoxystearate,
d) from 3% to 10% of water, and
e) a sufficient amount, to reach 100% of the weight of the composition, of at least one physiologically acceptable nonaqueous solvent capable of dissolving the release matrix, the active principle and the transcutaneous absorption promoter, and also of being rapidly eliminated by evaporation on contact with the skin, chosen from dichloromethane, ethanol, isopropanol or ethyl acetate.

25. A sprayable pharmaceutical composition according to claim 24 wherein:
the cellulosic polymer or copolymer is ethylcellulose,
the active principle is ibuprofen,
the transcutaneous absorption promoter is 2-ethylhexyl 2-ethylhexanoate, and
the physiologically acceptable solvent is isopropanol.

26. A sprayable pharmaceutical composition according claim 1 which is applied by direct spraying, without the aid of a compressed or liquefied propellant gas.

27. A sprayable pharmaceutical composition according to claim 1 additionally containing an aromatizing fraction consisting of one or more aromatizing compounds.

28. A sprayable pharmaceutical composition according to claim 27 wherein the aromatizing fraction is present at concentrations not exceeding 5% of the weight of the composition.

29. A sprayable pharmaceutical composition according to claim 28 wherein the aromatizing fraction consists of levomenthol.

30. A sprayable pharmaceutical composition according to claim 25 which is applied by direct spraying, without the aid of a compressed or liquefied propellant gas.

31. A sprayable pharmaceutical composition according to claim 25 additionally containing an aromatizing fraction consisting of one or more aromatizing compounds.

32. A sprayable pharmaceutical composition according to claim 31 wherein the aromatizing fraction is present at concentrations not exceeding 5% of the weight of the composition.

33. A sprayable pharmaceutical composition according to claim 32 wherein the aromatizing fraction consists of levomenthol.

34. A sprayable pharmaceutical composition according to claim 25 comprising by weight:
0.5% ethylcellulose;
5% ibuprofen;
20% 2-ethylhexyl 2-ethylhexanoate
4% water; and
70.5% isopropanol.
